(19) United States
(12) Patent Application Publication
(10) Pub. No.: US 2007/0099212 A1
(43) Pub. Date: May 3, 2007

(54) Consecutive base single molecule sequencing

(76) Inventor: Timothy Harris, Ocean County, NJ (US)

Correspondence Address: GOODWIN PROCTER LLP;PATENT ADMINISTRATOR, EXCHANGE PLACE, BOSTON, MA, 02109-2881, US

(21) Appl. No.: 11/496,063

(22) Filed: Jul. 28, 2006

Related U.S. Application Data
(60) Provisional application No. 60/703,777, filed on Jul. 28, 2005.

(51) Int. Cl. C12Q 1/68; C12M 1/34; C07H 21/04

(52) U.S. Cl. 435/6; 435/287.2; 536/25.32

(57) ABSTRACT

The invention provides methods for sequencing polynucleotide molecules using single molecule sequencing techniques, where a plurality of labeled nucleotides are incorporated consecutively into an individual primer molecule.

### RELATED APPLICATIONS

This application claims priority to U.S. Ser. No. 60/703,777 filed Jul. 28, 2005 and hereby incorporated by reference in its entirety.

### FIELD OF THE INVENTION

The invention relates generally to methods and materials for long-run consecutive base single molecule sequencing with high accuracy with respect to a reference sequence.

### BACKGROUND OF THE INVENTION

Completion of the human genome has paved the way for important insights into biologic structure and function and has given rise to inquiry into genetic differences between individuals, as well as differences within an individual, as the basis for differences in biological function and dysfunction. For example, single nucleotide differences between individuals, called single nucleotide polymorphisms (SNPs), are responsible for dramatic phenotypic differences. Those differences can be outward expressions of phenotype or can involve the likelihood that an individual will get a specific disease or how that individual will respond to treatment. Moreover, subtle genomic changes have been shown to be responsible for the manifestation of genetic diseases, such as cancer. A true understanding of the complexities in either normal or abnormal function may require large amounts of specific sequence information.

An understanding of cancer also requires an understanding of genomic sequence complexity. Cancer is a disease that is rooted in heterogeneous genomic instability. Most cancers develop from a series of genomic changes, some subtle and some significant, that occur in a small subpopulation of cells. Knowledge of the sequence variations that lead to cancer will lead to an understanding of the etiology of the disease, as well as ways to treat and prevent it.

The ability to perform high-resolution sequencing is a necessary first step towards understanding genomic complexity. Various approaches to nucleic acid sequencing exist. One conventional sequencing method consists of chain termination and gel separation, essentially as described by Sanger et al., Proc. Natl. Acad. Sci., 74(12): 5463-67 (1977). That method relies on the generation of a mixed population of nucleic acid fragments representing terminations at each base in a sequence. The fragments are then run on an electrophoretic gel and the sequence is revealed by the order of fragments in the gel. Another conventional bulk sequencing method relies on chemical degradation of nucleic acid fragments. See, Maxam et al., Proc. Natl. Acad. Sci., 74: 560-564 (1977). Finally, methods have been developed based upon sequencing by hybridization. See, e.g., Drmanac, et al., Nature Biotech., 16: 54-58 (1998).

The conventional sequencing methods described above are representative of bulk sequencing techniques. However, bulk sequencing is not useful for the identification of subtle or rare nucleotide changes. Cloning, amplification, and electrophoresis steps obscure useful information regarding individual nucleotides. As such, research has evolved toward methods for rapid sequencing, such as single molecule sequencing technologies. The ability to sequence and gain information from single molecules obtained from an individual patient is the next milestone for genomic sequencing.

There have been many proposals for single-molecule sequencing of DNA. Generally, those techniques involve the interaction of particular proteins with DNA or the use of ultra high resolution scanned probe microscopy. See, e.g., Rigler, et al., J. Biotech, 86(3): 161 (2001); Goodwin, P. M., et al., Nucleosides & Nucleotides, 16(5-6): 543-550 (1997); Howorka, S., et al., Nat. Biotech., 19(7): 636-639 (2001); Meller, A., et al., PNAS 97(3): 1079-1084 (2000); (2000); Driscoll, R. J., et al., Nature, 346(6281): 294-296(1990). Recently, Braslavasky, et al. have reported single molecule sequencing but only with spaces between the incorporated labeled nucleotides. See Braslavsky, et al., PNAS, 100:3960-3964 (2003). In other words, Braslavsky did not report consecutive base sequencing. Moreover, that paper reports that only 4 non-consecutive nucleotides were incorporated in the context of a much larger potential sequence run.

The present invention provides methods and materials for long-run consecutive base single molecule sequencing with high accuracy with respect to a reference sequence.

### SUMMARY OF THE INVENTION

The invention provides single molecule nucleic acid sequencing in which labeled nucleotides are incorporated consecutively in sequencing-by-synthesis reaction. Methods of the invention provide sequencing-by-synthesis conducted on single, optically-isolated nucleic acid duplexes attached to a surface and may combine surface preparation, oligonucleotide attachment, effective imaging and/or removal of incorporated labels in order to produce long sequence reads with high accuracy.

In one embodiment, a method for single molecule nucleic acid sequencing is provided comprising covalently bonding to a surface individually optically resolvable duplexes comprising a nucleic acid template and a primer hybridized thereto; conducting a template-dependent sequencing reaction mediated by a polymerase to extend primers of plural said optically resolvable duplexes by at least three consecutive optically labeled nucleotides; and detecting optically, by observation at known positions on said surface, the addition of labeled nucleotides to individual said duplexes thereby to determine the sequence of at least three bases of respective said templates with an accuracy of at least 70% with respect to a reference sequence. The covalent bonding may be conducted, for example, by coating said surface with an coating agent which covalently bonds with said template or said primer, the method comprising the additional step of exposing said coated surface to a blocking agent which inhibits non-specific binding thereto.

In some embodiments, the primer portion of said duplex is bonded to said surface. In other embodiments, the template portion of said duplex is bonded to said surface.

Coating agents, in an embodiment, comprise epoxide moities. For example, the template portion and the primer portion of a duplex may be bonded via an amine linkage to said epoxide. Blocking agents may be selected from the group consisting of water, a sulfite, an amine, a detergent, and a phosphate. In an embodiment, the blocking agent is Tris[hydroxymethyl]aminomethane.

The sequence determination may have an accuracy between about 75% and about 90%, or between about 90% and about 99%, or may be greater than about 99%.

Labeled nucleotides may be is labeled with an optically detectable label, for example a fluorescent group. In some embodiments, a fluorescent label is selected from the group consisting of fluorescein, rhodamine, cyanine, Cy5, Cy3, BODIPY, alexa, and derivatives thereof.

Methods contemplated herein may further comprise the additional step of compiling a linear sequence based upon sequential nucleotide incorporations in each member of said plurality of duplexes. Such a step may further comprise the additional step of aligning said linear sequence with a reference sequence.

In some embodiments, a coated surface including an epoxide is derivatized with one half of a binding pair and said template or said primer is derivatized with the other of said binding pair. Such binding pairs may be an antigen/antibody binding pair, or a biotin/streptavidin pair.

In another embodiment, a method of sequencing a nucleic acid template is provided comprising (a) exposing a nucleic acid template hybridized to a primer having a 3' end to (i) a polymerase which catalyzes nucleotide additions to the primer, and (ii) a labeled nucleotide under conditions to permit the polymerase to add the labeled nucleotide to the primer; (b) detecting the labeled nucleotide added to the primer in step (a); (c)removing the label from the labeled nucleotide; and repeating steps (a), (b) and (c) thereby to determine the sequence of at least three bases of respective said templates with an accuracy of at least 70% with respect to a reference sequence. Step (d) may be repeated at least four, ten or more times. In some embodiments, the template may be immobilized to a solid support, for example in an array at a density sufficient to detect and sequence single molecules individually.

In a preferred method of the invention, a nucleic acid duplex comprising a template and a primer hybridized thereto are attached to a surface that has low native fluorescence, e.g. does not substantially fluoresce. A preferred surface for conducting methods of the invention is an epoxide surface on a glass or fused silica slide or coverslip. However, any surface that has low native fluorescence and/or is capable of binding nucleic acids may be useful in the invention. Other surfaces include, but are not limited to, Teflon, polyelectrolyte multilayers, and others. In some embodiments, the surface may be passivated with a reagent that occupies portions of the surface that might, absent passivation, fluoresce. Passivation reagents, or blocking agents include amines, phosphate, water, sulfates, detergents, and other reagents that reduce native or accumulating surface fluorescence.

In some embodiments, the primer is part of an optically isolated substrate-bound duplex comprising a nucleic acid template having the primer hybridized thereto. The duplex may bound to the substrate such that the duplex is individually optically resolvable on the substrate.

In a preferred embodiment, the duplex may comprise a label, such as an optically-detectable label, that may be used to determine the position of individual duplex molecules on the surface. Once duplex positions are ascertained, the surface may be exposed to a labeled nucleotide triphosphate in the presence of a polymerase, allowing template strands that contain the complement of the labeled nucleotide immediately adjacent the 3′ terminus of the primer to incorporate the added nucleotide. After a wash step to remove unincorporated nucleotide, the surface may be imaged in order to determine which duplex positions have incorporated a labeled nucleotide. After imaging, label is optionally removed or silenced and the cycle may be repeated by adding another labeled nucleotide. The data set produced may be a stack of image data that shows the linear sequence of nucleotides incorporated at each of the individual duplex positions identified on the surface, after a sufficient or desired number of nucleotides (determined by the desired read length as discussed below) has been exposed to the surface-bound templates.

Preferred methods for single molecule sequencing of nucleic acid templates comprise conducting a template-dependent sequencing reaction in which multiple labeled nucleotides are incorporated consecutively into a primer such that the accuracy of the resulting sequence is at least 70% with respect to a reference sequence, between about 75% and about 90% with respect to a reference sequence, or between about 90% and about 99% with respect to a reference sequence. Preferably, the accuracy of the resulting sequence can be greater than about 99% with respect to a reference sequence. The reference sequence can be, for example, the sequence of the template nucleic acid molecule, if known, or the sequence of the template obtained by other sequencing methods, or the sequence of a corresponding nucleic acid from a different source, for example from a different individual of the same species or the same gene from a different species.

As described herein, a plurality of labeled nucleotides are incorporated consecutively into one or more individual primer molecules. After each incorporation, the label of the nucleotide may be removed. In some embodiments, at least three consecutive nucleotides, each initially comprising an optically-detectable label, are incorporated into an individual primer molecule. In other embodiments, at least 5, at least 10, at least 20, at least 30, at least 50, at least 100, at least 500, at least 1000 or at least 10000 consecutive nucleotides, each nucleotide initially comprising an optically-detectable label are incorporated into an individual primer molecule.

Sequencing may be accomplished by presenting one or more labeled nucleotides in the presence of a polymerase under conditions that promote complementary base incorporation in the primer. In an embodiment, one base at a time (per cycle) is added and all bases have the same label. There may be a wash step after each incorporation cycle. Once the surface is imaged, the label is either neutralized without removal or removed from incorporated nucleotides. After the completion of a predetermined number of cycles of base addition, the linear sequence data for each individual duplex is compiled, for example, by using the imaging data together with an appropriate algorithm. Such algorithms are available for sequence compilation and alignment as discussed below.

Nucleic acid template molecules include deoxyribonucleic acid (DNA) and/or ribonucleic acid (RNA). Nucleic acid template molecules can be isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. Biological samples of the present invention include viral particles or preparations. Nucleic acid template molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid template molecules may also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, or genomic DNA.

Nucleic acid obtained from biological samples typically is fragmented to produce suitable fragments for analysis. In one embodiment, nucleic acid from a biological sample is fragmented by sonication. Nucleic acid template molecules can be obtained as described in U.S. Patent Application 2002/0190663 A1, published Oct. 9, 2003, the teachings of which are incorporated herein in their entirety. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, individual nucleic acid template molecules can be from about 5 bases to about 20 kb. Nucleic acid molecules may be single-stranded, double-stranded, or double-stranded with single-stranded regions (for example, stem- and loop-structures).

Methods according to the invention provide de novo sequencing, re-sequence, DNA fingerprinting, polymorphism identification, for example single nucleotide polymorphisms (SNP) detection, as well as applications for genetic cancer research. Applied to RNA sequences, methods according to the invention also are useful to identify alternate splice sites, enumerate copy number, measure gene expression, identify unknown RNA molecules present in cells at low copy number, annotate genomes by determining which sequences are actually transcribed, determine phylogenic relationships, elucidate differentiation of cells, and facilitate tissue engineering. Methods according to the invention are also useful to analyze activities of other biomacromolecules such as RNA translation and protein assembly.

Other aspects and advantages of the invention are apparent to the skilled artisan upon consideration of the following drawings, detailed description of the invention and example.

### BRIEF DESCRIPTION OF THE DRAWINGS

FIG. 1 depicts exemplary nucleotide analogs including cleavable labels.

FIG. 2 is an exemplary schematic showing molecules viewed as an image stack.

FIG. 3 shows an exemplary imaging system of the present invention.

FIG. 4 shows an exemplary flow cell of the present invention.

FIG. 5 depicts a chart showing the accuracy of sequencing M13 using the methods of the present invention.

FIG. 6 is an exemplary schematic showing a passivated epoxide surface with attached nucleic acids.

### DETAILED DESCRIPTION

Single molecule sequencing according to the invention may be conducted, for example, by attaching template/primer duplex to an epoxide surface such that duplex was individually optically resolvable (i.e., resolvable from other duplexes on the surface). Parallel sequencing-by-synthesis reactions may be conducted on the surface using optical detection of incorporated nucleotides followed by sequence compilation. Further, methods disclosed herein may be used for de novo sequencing or resequencing of a reference sequence. Partial sequencing can also be conducted using methods of the invention as will be apparent to those of ordinary skill in the art upon consideration of the disclosure herein.

In general, epoxide-coated glass surfaces can be used for direct amine attachment of templates, primers, or both. For example, amine attachment to the termini of template and primer molecules can be accomplished using terminal transferase as described below. In some embodiments, primer molecules can be custom-synthesized to hybridize to templates for duplex formation. In a preferred embodiment, as described below, template fragments are polyadenylated and a complementary poly(dT) oligo is used as the primer. In this way, surfaces having previously-bound universal primers can be prepared for sequencing heterogeneous fragments obtained from genomic DNA or RNA.

In a preferred embodiment, nucleic acid template molecules are attached to a substrate (also referred to herein as a surface) and subjected to analysis by single molecule sequencing as taught herein. Nucleic acid template molecules are attached to the surface at a density such that the template/primer duplexes are individually optically resolvable. Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

In one embodiment, a substrate may be coated to allow optimum optical processing and nucleic acid attachment. In other embodiments, substrates for use in the invention may be treated to reduce background noise. Exemplary coatings include epoxides and derivatized epoxides (e.g., with a binding molecule, such as streptavidin). Examples of substrate coatings include, vapor phase coatings of 3-aminopropyltrimethoxysilane, as applied to glass slide products, for example, from Molecular Dynamics, Sunnyvale, Calif.

A surface may also be treated to improve the positioning of attached nucleic acids (e.g., nucleic acid template molecules, primers, or template molecule/primer duplexes) for analysis. For example, hydrophobic substrate coatings and films may aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase template molecule binding to the substrate. As such, a surface according to the invention can be treated with one or more charge layers (e.g., a negative charge) to repel a charged molecule (e.g., a negatively charged labeled nucleotide).

For example, a substrate according to the invention can be treated with polyallylamine followed by polyacrylic acid to form a polyelectrolyte multilayer. The carboxyl groups of such a polyacrylic acid layer are negatively charged and thus may repel negatively charged labeled nucleotides, improving the positioning of the label for detection. Coatings or films that may be used with a substrate should be able to withstand subsequent treatment steps (e.g., photoexposure, boiling, baking, soaking in warm detergent-containing liquids, and the like) without substantial degradation or disassociation from the substrate.

Various methods can be used to anchor or immobilize the nucleic acid template molecule to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11:107-115, 1986. A preferred attachment is direct amine bonding of a terminal nucleotide of the template or the primer to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

Single molecule sequencing according to this disclosure may combine sample preparation, surface preparation and oligo attachment, imaging, and/or analysis in order to achieve high-throughput sequence information. For example, optically-detectable labels may be attached to primers that are attached directly to an epoxide surface. Individual primer molecules can then be imaged in order to establish their positions on the surface. Individual nucleotides containing an optical label can then be added in the presence of polymerase for incorporation into the 3′ end of the primer at a location in which the added nucleotide is complementary to the next-available nucleotide on the template immediately 5′ (on the template) of the 3′ terminus of the primer. Unbound nucleotide may then be washed out. In some embodiments, a scavenger may be added. The surface that includes incorporated labeled nucleotides may then be imaged, for example, detecting an optical signal at a position previously noted to contain a single duplex (or primer) is counted as an incorporation event. In some embodiments, the nucleotide label can then removed and any remaining linker may be capped before the system is again washed.

Any polymerizing enzyme may be used in the invention. A preferred polymerase is Klenow with reduced exonuclease activity. Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Known conventional DNA polymerases useful in the invention include, but are not limited to, Pyrococcus furiosus (Pfu) DNA polymerase (Lundberg) et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9° Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerise (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J Biol. Chem. 256:3112), and archaeal DP II/DP2 DNA polymerase II (Cann et al., 1998, Proc Natl Acad. Sci. USA 95:14250->5).

Other DNA polymerases include, but are not limited to, ThermoSequenase®, 9° Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347(1975)).

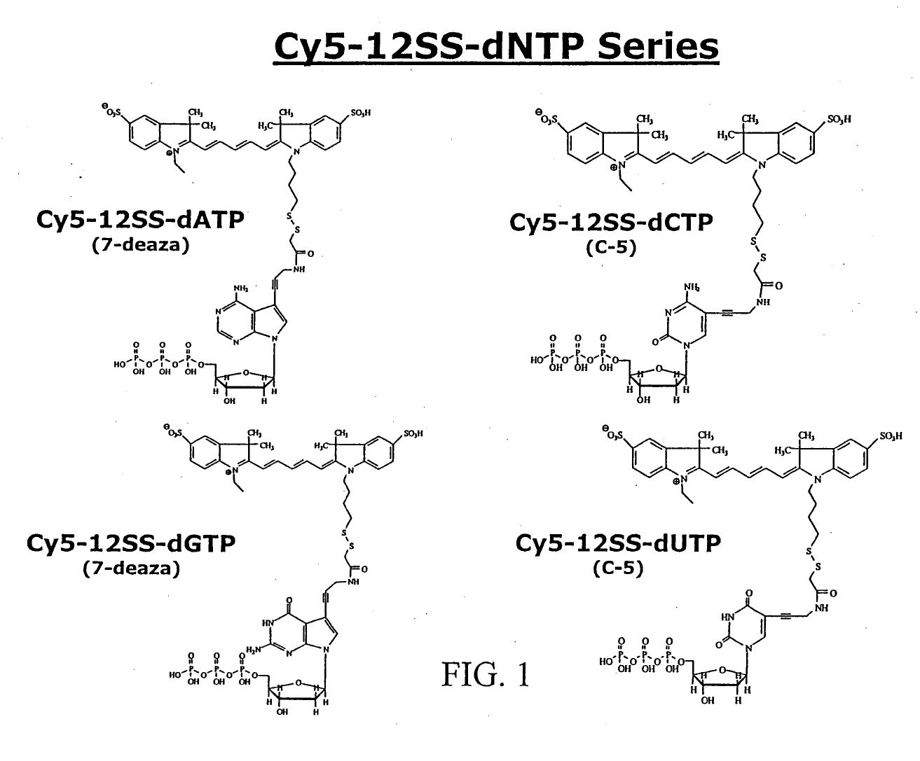

The cycle may be repeated with remaining nucleotides. In a particular embodiment of the invention, all four nucleotides are added in each cycle, with each nucleotide containing a detectable label. In a highly-preferred embodiment of the invention, the label attached to added nucleotides is an optically detectable label, for example, a fluorescent label. Examples of fluorescent labels include, but are not limited to, 4-acetamido-4′-isothiocyanatostilbene2,2′disulfonic acid; acridine and derivatives: acridine, acridine isothiocyanate; 5-(2′aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-I-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; MD 800; La Jolta Blue; phthalo cyanine; and naphthalo cyanine. Preferred fluorescent labels are cyanine-3 and cyanine-5. FIG. 1 shows the structure of cyanine-5 attached to the four common nucleotides. Labels other than fluorescent labels are contemplated by the invention, including other optically-detectable labels. Exemplary cleavable labels are shown attached to nucleotides in FIG. 1.

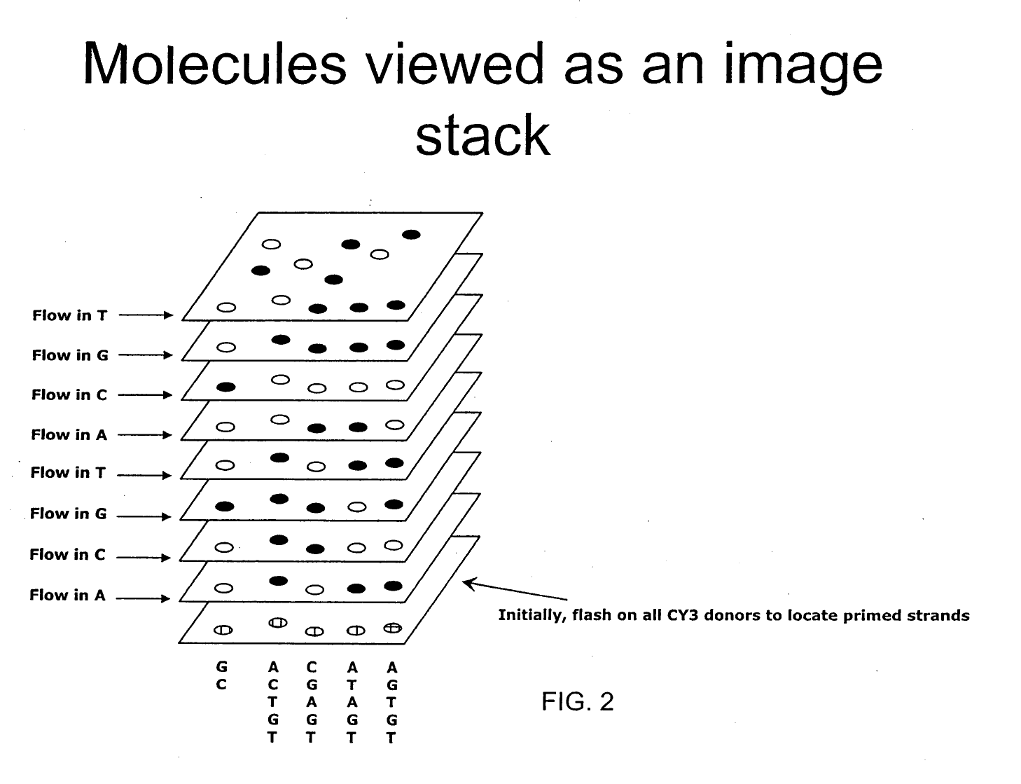

A full-cycle is conducted as many times as necessary to complete sequencing of a desired length of template. Once the desired number of cycles is complete, the result is a stack of images as shown in FIG. 2 represented in a computer database. As FIG. 2 shows, for each spot on the surface that contained an initial individual duplex, there will be a series of light and dark image coordinates, corresponding to whether a base was incorporated in any given cycle. For example, if the template sequence was TACGTACG and nucleotides were presented in the order CAGU(T), then the duplex would be “dark” (i.e., no detectable signal) for the first cycle (presentation of C), but would show signal in the second cycle (presentation of A, which is complementary to the first T in the template sequence). The same duplex would produce signal upon presentation of the G, as that nucleotide is complementary to the next available base in the template, C. Upon the next cycle (presentation of U), the duplex would be dark, as the next base in the template is G. Upon presentation of numerous cycles, the sequence f the template would be built up through the image stack. The sequencing data are then fed into an aligner as described below for resequencing, or are compiled for de novo sequencing as the linear order of nucleotides incorporated into the primer.

There are numerous alternatives to practice of the invention. For example, while a primer may be attached via a direct amine attachment to an epoxide surface, in an alternative embodiment, the template may form a duplex and may be attached first (i.e., a duplex was formed first and then attached to the surface). In another alternative embodiment, an epoxide surface may be functionalized with one member of a binding pair, the other member of the binding pair being attached to the template, primer, or both for attachment to the surface. For example, the surface can be functionalized with stretptavidin with biotin attached to the termini of either the template, the primer, or both.

In another embodiment of the invention, fluorescence resonance energy transfer (FRET) is used to generate one or more signals from incorporated nucleotides in single molecule sequencing of the invention. FRET can be conducted as described in Braslavsky, et al., 100 PNAS: 3960-64 (2003), incorporated by reference herein. In one embodiment, a donor fluorophore is attached to the primer portion of the duplex and an acceptor fluorophore is attached to a nucleotide to be incorporated. In other embodiments, donors are attached to the template, the polymerase, or the substrate in proximity to a duplex. In any case, upon incorporation, excitation of the donor produces a detectable signal in the acceptor to indicate incorporation.

In another embodiment of the invention, nucleotides presented to the surface for incorporation into a surface-bound duplex comprise a reversible blocker. A preferred blocker is attached to the 3′ hydroxyl on the sugar moiety of the nucleotide. For example an ethyl cyanine (—OH—CH2CH2CN) blocker, which is removed by hydroxyl addition to the sample, is a useful removable blocker. Other useful blockers include fluorophores placed at the 3′ hydroxyl position, and chemically labile groups that are removable, leaving an intact hydroxyl for addition of the next nucleotide, but that inhibit further polymerization before removal.

In another embodiment, individually optically resolvable complexes comprising polymerase and a target nucleic acid are oriented with respect to each other for complementary base addition in a zero mode waveguide. In one embodiment, an array of zero-mode waveguides comprising subwavelength holes in a metal film is used to sequence DNA or RNA at the single molecule level. A zero-mode waveguide is one having a wavelength cut-off above which no propagating modes exist inside the waveguide. Illumination decays rapidly incident to the entrance to the waveguide, thus providing very small observation volumes. In one embodiment, the waveguide consists of small holes in a thin metal film on a microscope slide or coverslip. Polymerase is immobilized in an array of zero-mode waveguides. The waveguide is exposed to a template/primer duplex, which is captured by the enzyme active site. Then a solution containing a species of fluorescently-labeled nucleotide is presented to the waveguide, and incorporation is observed after a wash step as a burst of fluorescence.

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is non-denaturing, can act to solubilize the sample. Detergents may be ionic or nonionic. Examples of nonionic detergents include triton, such as the Triton® X series (Triton® X-100 t-Oct-C₆H₄-(OCH₂-CH₂)XOH, x=9-10, Triton® X-100R, Triton(& X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, ndodecyl beta-D-maltoside (DDM), NP-40 nonylphenyl polyethylene glycol, C12E8 (octaethylene glycol n-dodecyl monoether), hexaethyleneglycol mono-n-tetradecyl ether (C 14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant. Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid.

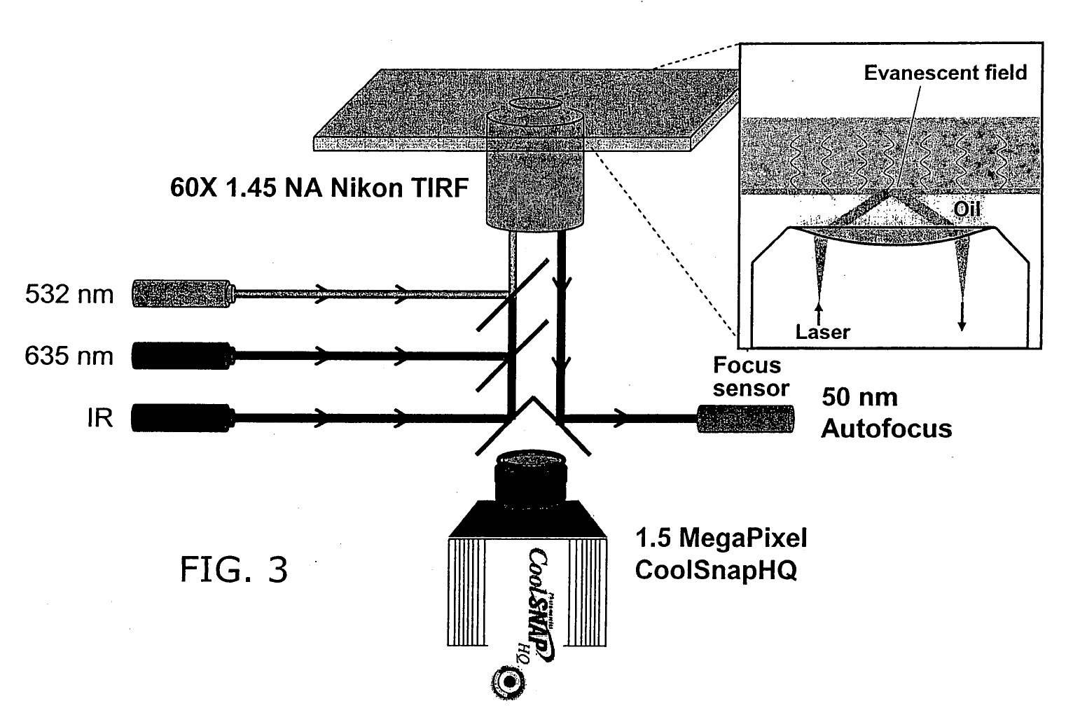

The imaging system to be used in the invention can be any system that provides sufficient illumination of the sequencing surface at a magnification such that single fluorescent molecules can be resolved. The imaging system used in the example described below is shown in FIG. 3. In general, the system comprises three lasers, one that produces “green” light, one that produces “red” light, and in infrared laser that aids in focusing. The beams are transmitted through a series of objectives and mirrors, and focused on the image as shown in FIG. 3. Imaging is accomplished with an inverted Nikon TE-2000 microscope equipped with a total internal reflection objective (Nikon).

However, any detection method may be used that is suitable for the type of nucleotide label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (STM) and the atomic force microscope (AFM). For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass.), Genix Technologies (Waterloo, Ontario, Canada; on the World Wide Web at confocal.com), and Applied Precision Inc. Such detection methods may particularly useful to achieve simultaneous scanning of multiple attached template nucleic acids.

Further exemplary approaches that may be used to detect incorporation of fluorescently-labeled nucleotides into a single nucleic acid molecule include optical setups that may include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection hybridization patterns from laser-activated fluorescence using a microscope equipped with a camera, for example a CCD camera (e.g., Model TE/CCD512SF, Princeton Instruments, Trenton, N.J.) with suitable optics (e.g., Ploem, in Fluorescent and Luminescent Probes for Biological Activity Mason, T.G. Ed., Academic Press, Landon, pp. 1-11 (1993), such as described in Yershov et al., Proc. Natl. Acad.Sci. 93:4913 (1996), or may be imaged by TV monitoring. Suitable photon detection systems may include photodiodes.

For example, an intensified charge couple device (ICCD) camera can be used for detecting or imaging individual fluorescent dye molecules in a fluid near a surface. In some embodiments, an ICCD optical setup may be used to acquire a sequence of images (movies) of fluorophores.

Some embodiments of the present invention may use TIRF microscopy for two-dimensional imaging. TIRF microscopy uses totally internally reflected excitation light and is well known in the art. See, e g., the World Wide Web at www.coolscope.com/eng/page/products/tirf.aspx. In certain embodiments, detection is carried out using evanescent wave illumination and total internal reflection fluorescence microscopy. A n evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors.

In resequencing, a preferred embodiment for sequence alignment may compare sequences obtained to a database of reference sequences of the same length, or within 1 or 2 bases of the same length, from the target in a look-up table format. In a preferred embodiment, the look-up table contains exact matches with respect to the reference sequence and sequences of the prescribed length or lengths that have one or two errors (e.g., 9-mers with all possible 1-base or 2-base errors). The obtained sequences are then matched to the sequences on the look-up table and given a score that reflects the uniqueness of the match to sequence(s) in the table. The obtained sequences are then aligned to the reference sequence based upon the position at which the obtained sequence best matches a portion of the reference sequence.

### EXAMPLE

The 7249 nucleotide genome of the bacteriophage M13mp18 was sequenced using single molecule methods of the invention. Purified, single-stranded viral M13mp18 genomic DNA was obtained from New England Biolabs. Approximately 25 μg of M13 DNA was digested to an average fragment size of 40 by with 0.1 U Dnase I (New England Biolabs) for 10 minutes at 37° C. Digested DNA fragment sizes were estimated by running an aliquot of the digestion mixture on a precast denaturing (TBE-Urea) 10% polyacrylamide gel (Novagen) and staining with SYBR Gold (Invitrogen/Molecular Probes). T he DNase I-digested genomic DNA was filtered through a YM10 ultrafiltration spin column (Millipore) to remove small digestion products less than about 30 nt. Approximately 20 pmol of the filtered DNase I digest was then polyadenylated with terminal transferase according to known methods (Roychoudhury, R and Wu, R. 1980, Terminal transferase-catalyzed addition of nucleotides to the 3′ termini of DNA. Methods Enzymol. 65(1):43-62.). The average dA tail length was 50+/−5 nucleotides. Terminal transferase was then used to label the fragments with Cy3-dUTP. Fragments were then terminated with dideoxyTTP (also added using terminal transferase). The resulting fragments were again filtered with a YM 10 ultrafiltration spin column to remove free nucleotides and stored in ddH2O at −20° C.

Epoxide-coated glass slides were prepared for oligo attachment. Epoxide-functionalized 40 mm diameter #1.5 glass cover slips (slides) were obtained from Erie Scientific (Salem, NH). The slides were preconditioned by soaking in 3×SSC for 15 minutes at 37° C.

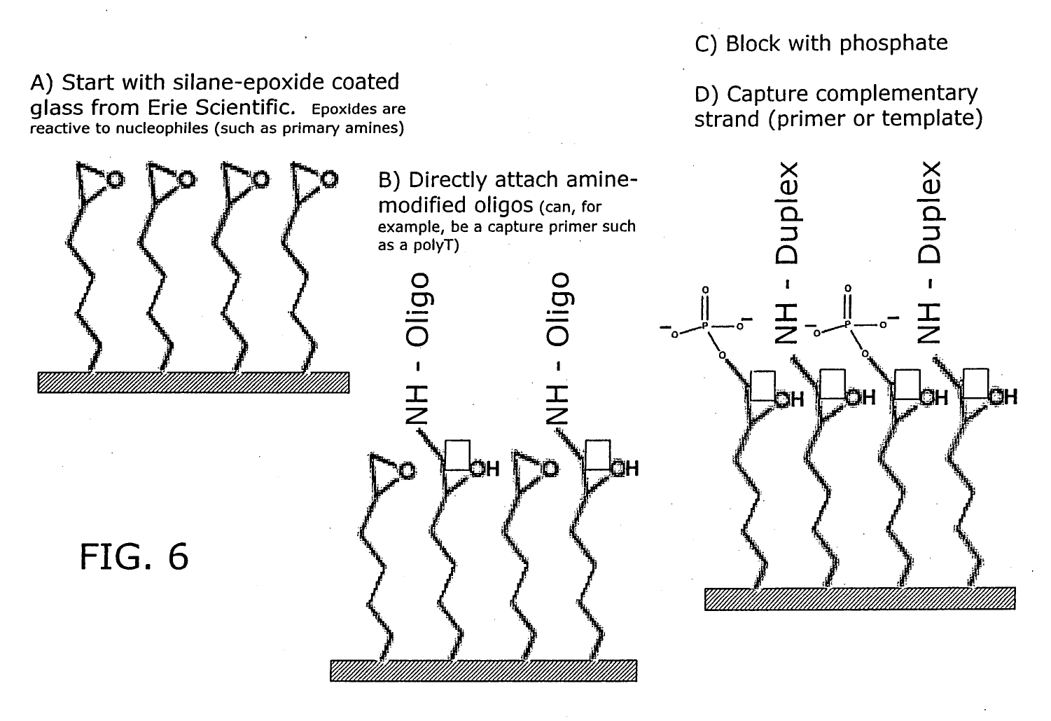

Next, a 500 pM aliquot of 5′ aminated polydT(50) (polythymidine of 50 bp in length with a 5′ terminal amine) was incubated with each slide for 30 minutes at room temperature in a volume of 80 ml. The resulting slides had poly(dT50) primer attached by direct amine linkage to the epoxide. The slides were then treated with phosphate (1 M) for 4 hours at room temperature in order to passivate the surface. Slides were then stored in polymerase rinse buffer (20 mM Tris, 100 mM NaCl, 0.001% Triton X-100, pH 8.0) until they were used for sequencing. A schematic of a passivated epoxide surface with attached oligos is shown in FIG. 6.

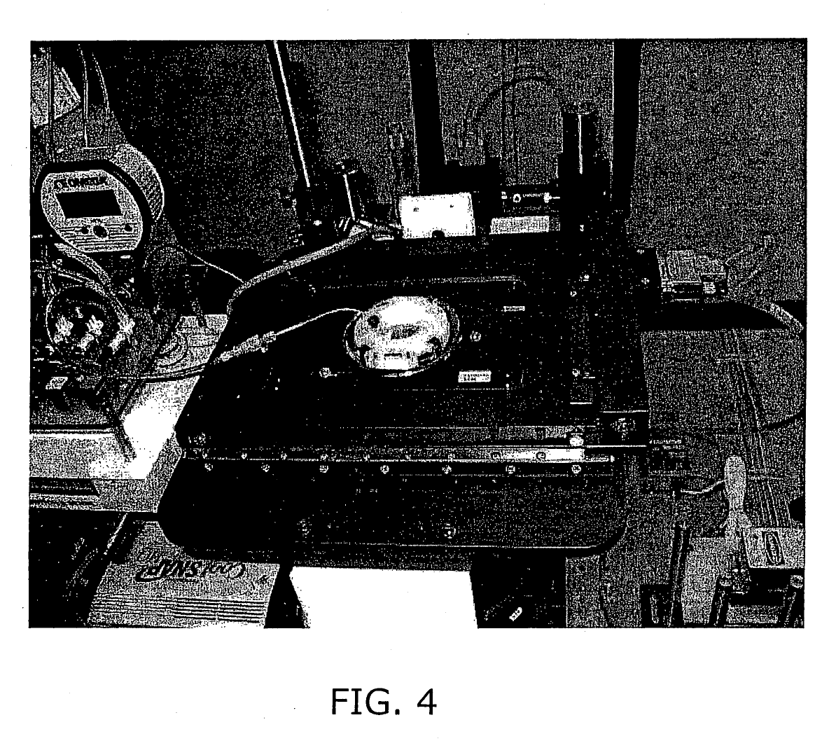

For sequencing, the slides were placed in a modified FCS2 flow cell (Bioptechs, Butler, Pa.) using a 50 μm thick gasket, as shown in FIG. 4. The flow cell was placed on a movable stage that is part of a high-efficiency fluorescence imaging system built around a Nikon TE-2000 inverted microscope equipped with a total internal reflection (TIR) objective. A schematic of the optical setup is shown in FIG. 3. The slide was then rinsed with HEPES buffer with 100 MM NaCl and equilibrated to a temperature of 50° C. An aliquot of the M13 template fragments described above was diluted in 3×SSC to a final concentration of 1.2 nM. A 100 ul aliquot was placed in the flow cell and incubated on the slide for 15 minutes. After incubation, the flow cell was rinsed with 1×SSC/HEPES/0.1%SDS followed by HEPES/NaCl. A passive vacuum apparatus was used to pull fluid across the flow cell. The resulting slide contained M13 template/olig(dT) primer duplex. The temperature of the flow cell was then reduced to 37° C. for sequencing and the objective was brought into contact with the flow cell.

For sequencing, cytosine triphosphate, guanidine triphosphate, adenine triphosphate, and uracil triphosphate, each having a cyanine-5 label (at the 7-deaza position for ATP and GTP and at the C5 position for CTP and UTP (PerkinElmer)) were stored separately in buffer containing 20 mM Tris-HCl, pH 8.8, 10 mM MgSO₄, 10 MM (NH₄)₂, 10 mM HCl, and 0.1% Triton X-100, and 100U Kienow exo⁻ polymerase (NEN). Sequencing proceeded as follows.

First, initial imaging was used to determine the positions of duplex on the epoxide surface. The Cy3 label attached to the M13 templates was imaged by excitation using a laser tuned to 532 nm radiation (Verdi V-2 Laser, Coherent, Inc., Santa Clara, Calif.) in order to establish duplex position. For each slide only single fluorescent molecules were imaged in this step were counted. Imaging of incorporated nucleotides as described below was accomplished by excitation of a cyanine-5 dye using a 635 nm radiation laser (Coherent). 5 uM Cy5CTP was placed into the flow cell and exposed to the slide for 2 minutes. After incubation, the slide was rinsed in 1×SSC/15 mM HEPES/0.1% SDS/pH 7.0 (“SSC/HEPES/SDS”) (15 times in 60 ul volumes each, followed by 150 mM HEPES/150 mM NaCl/pH 7.0 (“HEPES/NaCl”) (10 times at 60 ul volumes). An oxygen scavenger containing 30% acetonitrile and scavenger buffer (134 u1 HEPES/NaCl, 24 ul 100 mM Trolox in MES, pH6.1, 1- ul DABCO in MES, pH6.1, Sul 2M glucose, 20 ul Nal (50 mM stock in water), and 4 ul glucose oxidase) was next added. The slide was then imaged (500 frames) for 0.2 seconds using an Inova3OlK laser (Coherent) at 647 nm, followed by green imaging with a Verdi V-2 laser (Coherent) at 532 nm for 2 seconds to confirm duplex position. The positions having detectable fluorescence were recorded. After imaging, the flow cell was rinsed 5 times each with SSC/HEPES/SDS (60 ul) and HEPES/NaCl (60 ul). Next, the cyanine-5 label was cleaved off incorporated CTP by introduction into the flow cell of 50 mM TCEP for 5 minutes, after which the flow cell was rinsed 5 times each with SSC/HEPES/SDS (60 ul) and HEPES/NaCl (60 ul). The remaining nucleotide was capped with 50 mM iodoacetamide for 5 minutes followed by rinsing 5 times each with SSC/HEPES/SDS (60 ul) and HEPES/NaCl (60 ul). The scavenger was applied again in the manner described above, and the slide was again imaged to determine the effectiveness of the cleave/cap steps and to identify nonincorporated fluorescent objects.

The procedure described above was then conducted 100 nM Cy5dATP, followed by 100 nM Cy5dGTP, and finally 500 nM Cy5dUTP. The procedure (expose to nucleotide, polymerase, rinse, scavenger, image, rinse, cleave, rinse, cap, rinse, scavenger, final image) was repeated exactly as described for ATP, GTP, and UTP except that Cy5dUTP was incubated for 5 minutes instead of 2 minutes. Uridine was used instead of thymidine due to the fact that the Cy5 label was incorporated at the position normally occupied by the methyl group in thymidine triphosphate, thus turning the dTTP into dUTP. In all 64 cycles (C, A, G, U) were conducted as described in this and the preceding paragraph.

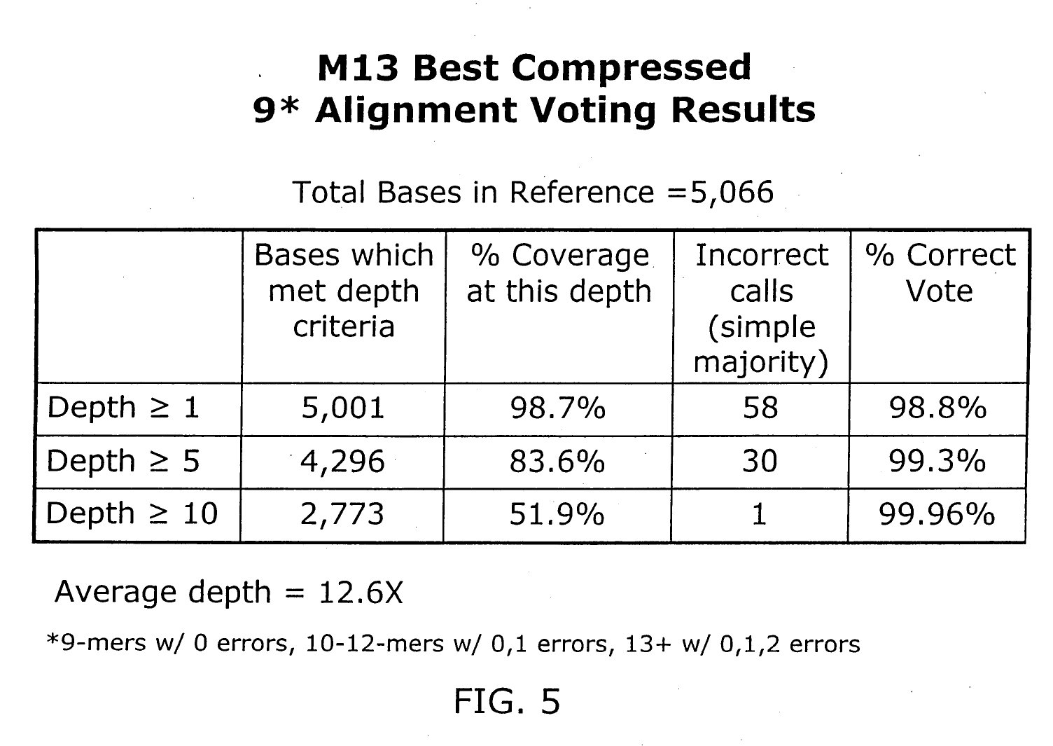

Once 64 cycles were completed, the image stack data (i.e., the single molecule sequences obtained from the various surface-bound duplex) were aligned to the M13 reference sequence. The image data obtained was compressed to collapse homopolymeric regions. Thus, the sequence “TCAAAGC” would be represented as “TCAGC” in the data tags used for alignment. Similarly, homopolymeric regions in the reference sequence were collapsed for alignment. The results are shown in FIG. 5. The sequencing protocol described above resulted in an aligned M13 sequence with an accuracy of between 98.8% and 99.96% (depending on depth of coverage). The individual single molecule sequence read lengths obtained ranged from 2 to 33 consecutive nucleotides with about 12.6 consecutive nucleotides being the average length. The number of correct bases over the entire length of the M13 sequence and the percent correct base calls (accuracy) are shown in FIG. 5.

The alignment algorithm matched sequences obtained as described above with the actual M13 linear sequence. Placement of obtained sequence on M13 was based upon the best match between the obtained sequence and a portion of M13 of the same length, taking into consideration 0, 1, or 2 possible errors. All obtained 9-mers with 0 errors (meaning that they exactly matched a 9-mer in the M13 reference sequence) were first aligned with M13. Then 10-, 11-, and 12-mers with 0 or 1 error were aligned. Finally, all 13-mers or greater with 0, 1, or 2 errors were aligned. This gave the alignment shown in FIG. 5. As shown in that Figure, at a coverage depth of greater than or equal to 1, 5,001 based of the 5,066 base M13 genome were covered at an accuracy of 98.8%. Similarly, at a coverage depth of greater than or equal to 5, 83.6% of the genome was covered at an accuracy of 99.3%, and at a depth of greater than or equal to 10, 51.9% of the genome was covered at an accuracy of 99.96%. The average coverage depth was 12.6 nucleotides.

The sequence tags obtained from the fractionated M13 DNA are shown in Table I and Table II in the files entitled TABLE I COMPRESSED M13 SEQUENCE DATA.txt, created Jul. 28, 2005, 661 kB, and TABLE II UNCOMPRESSED M13 SEQUENCE DATA.txt, 739 kB, created Jul. 28, 2005 both included in the accompanying compact disk and which forms part of this disclosure, filed herewith and both incorporated by reference in their entirety. These results show that single molecule methods of the invention produced high consecutive read lengths and overall high accuracy against the M13 reference sequence.

All publications, patents, and patent applications cited herein are hereby expressly incorporated by reference in their entirety and for all purposes to the same extent as if each was so individually denoted.

While specific embodiments of the subject invention have been discussed, the above specification is illustrative and not restrictive. Many variations of the invention will become apparent to those skilled in the art upon review of this specification. Contemplated equivalents of the methods disclosed here include methods which otherwise correspond thereto, and which have the same general properties or result thereof, wherein one or more simple variations of substituents or components are made which do not adversely affect the characteristics of the methods of interest. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

We claim:

1. A method for single molecule nucleic acid sequencing, the method comprising:
covalently bonding to a surface individually optically resolvable duplexes comprising a nucleic acid template and a primer hybridized thereto;
conducting a template-dependent sequencing reaction mediated by a polymerase to extend primers of plural said optically resolvable duplexes by at least three consecutive optically labeled nucleotides; and
detecting optically, by observation at known positions on said surface, the addition of labeled nucleotides to individual said duplexes thereby to determine the sequence of at least three bases of respective said templates with an accuracy of at least 70% with respect to a reference sequence.

2. The method of claim 1, wherein the bonding is conducted by coating said surface with a coating agent which covalently bonds with said template or said primer, the method comprising the additional step of exposing said coated surface to a blocking agent which inhibits non-specific binding thereto.

3. The method of claim 2, wherein the primer portion of said duplex is bonded to said surface.

4. The method of claim 3, wherein the template portion of said duplex is bonded to said surface.

5. The method of claim 2, wherein said coating agent comprises epoxide moities.

6. The method of claim 5, wherein the template portion and the primer portion of said duplex is bonded via an amine linkage to said epoxide.

7. The method of claim 2, wherein said blocking agent is selected from the group consisting of water, a sulfite, an amine, a detergent, and a phosphate.

8. The method of claim 7, wherein said blocking agent is Tris[hydroxymethyl]aminomethane.

9. The method of claim 1 wherein the accuracy is between about 75% and about 90%.

10. The method of claim 1 wherein the accuracy is between about 90% and about 99%.

11. The method of claim 1 wherein the accuracy is greater than about 99%.

12. The method of claim 1 wherein said labeled nucleotide is labeled with an optically detectable label.

13. The method of claim 12, wherein said optically detectable label is a fluorescent label.

14. The method of claim 13, wherein said fluorescent label is selected from the group consisting of fluorescein, rhodamine, cyanine, Cy5, Cy3, BODIPY, alexa, and derivatives thereof.

15. The method of claim 1 comprising the additional step of compiling a linear sequence based upon sequential nucleotide incorporations in each member of said plurality of duplexes.

16. The method of claim 15 comprising the additional step of aligning said linear sequence with a reference sequence.

17. The method of claim 5, wherein said epoxide is derivatized with one half of a binding pair and said template or said primer is derivatized with the other of said binding pair.

18. The method of claim 17, wherein said binding pair is an antigen/antibody binding pair.

19. The method of claim 17, wherein said binding pair is biotin/streptavidin.

20. A method of sequencing a nucleic acid template comprising:
(a) exposing a nucleic acid template hybridized to a primer having a 3′ end to (i) a polymerase which catalyzes nucleotide additions to the primer, and (ii) a labeled nucleotide under conditions to permit the polymerase to add the labeled nucleotide to the primer;
(b) detecting optically, by observation at known positions on said surface the labeled nucleotide added to the primer in step (a);
(c) removing the label from the labeled nucleotide;
(d) repeating steps (a), (b) and (c) thereby to determine the sequence of at least three bases of respective said templates with an accuracy of at least 70% with respect to a reference sequence.

21. The method of claim 20, where step (d) is repeated at least four times.

22. The method of claim 20, wherein during step (a), the template is immobilized to a solid support.

23. The method of claim 20, wherein the template is immobilized in an array at a density sufficient to detect and sequence single molecules individually

24. A method for single molecule nucleic acid sequencing, the method comprising: conducting a template-dependent sequencing reaction in which multiple labeled nucleotides are incorporated consecutively into a primer portion of a substrate-bound duplex thereby producing a sequence, the substrate-bound duplex comprising a nucleic acid template and primer hybridized thereto, wherein said duplex is individually optically resolvable on said substrate, and wherein the accuracy of the resulting sequence is at least 70% with respect to a reference sequence.

25. The method of claim 24, wherein said substrate is glass.

26. The method of claim 25, wherein said glass is coated with an epoxide.

27. The method of claim 26, further comprising exposing said epoxide to a blocking agent capable of inhibiting non-specific binding of molecules to said epoxide.

28. The method of claim 27, wherein said blocking agent is selected from the group consisting of water, a sulfite, an amine, a detergent, and a phosphate.

29. The method of claim 28, wherein said detergent is Tris.

30. The method of claim 26, wherein said duplex is attached directly to said epoxide.

31. The method of claim 26, wherein the primer portion of said duplex is attached via an amine linkage to the epoxide.

32. The method of claim 26, wherein the template portion of said duplex is attached via an amine linkage to the epoxide.

33. The method of claim 32, wherein said epoxide is derivatized with a member of a binding pair and said duplex comprises another member of said binding pair.

34. The method of claim 33, wherein said binding pair is an antigen/antibody binding pair.

35. The method of claim 33, wherein said binding pair is biotin/streptavidin.

36. The method of claim 24, wherein said accuracy is between about 75% and about 90% with respect to said reference sequence.

37. The method of claim 24, wherein said accuracy is between about 90% and about 99% with respect to said reference sequence.

38. The method of claim 24, wherein said accuracy is greater than about 99% with respect to said reference sequence.

39. The method of claim 24, wherein said label is an optically-detectable label.

40. The method of claim 39, wherein said optically-detectable label is a fluorescent label.

41. The method of claim 40, wherein said fluorescent label is selected from the group consisting of fluorescein, rhodamine, cyanine, Cy5, Cy3, BODIPY, alexa, and derivatives thereof.

42. The method of claim 24, wherein said conducting step is performed on a plurality of duplexes on said substrate.

43. The method of claim 42, further comprising the step of compiling a linear sequence based upon sequential nucleotide incorporations in each member of said plurality of duplexes.

44. The method of claim 43, further comprising the step of aligning said linear sequence with a reference sequence.

45. The method of claim 44, wherein said plurality of duplexes comprises two or template portions having different sequences.

46. The method of claim 24, wherein the template-dependent sequencing reaction is performed in the absence of unlabeled nucleotides.
